SEQUENCE LISTING (1 sequence)

SEQ ID NO: 1 - protein, 262 aa, Bos taurus
LPVKPTSSGSSEEKQLNNKYPDAVAIWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKRKDKNKHSNLIESQENSKLSQEFHSLEDKLDLDHKSEEDKHLKIRISHELDSASSEVN

(19) United States
(12) Patent Application Publication
(10) Pub. No.: US 2010/0267139 A1
(43) Pub. Date: Oct. 21, 2010

(54) OSTEOPONTIN NANOPARTICLE SYSTEM FOR DRUG DELIVERY

(75) Inventors: Jørgen Kjems, Risskov (DK); Kenneth Alan Howard, Arhus (DK); Flemming Besenbacher, Arhus (DK); Morten Andreasen, Arhus (DK)

Correspondence Address: KNOBBE MARTENS OLSON & BEAR LLP, 2040 MAIN STREET, FOURTEENTH FLOOR, IRVINE, CA, 92614, US

(73) Assignee: AARHUS UNIVERSITET, Aarhus C (DK)

(21) Appl. No.: 12/669,112

(22) PCT Filed: Jul. 16, 2008
(86) PCT No.: PCT/DK2008/050179

§ 371 Date: Jul. 1, 2010

(30) Foreign Application Priority Data
Jul. 16, 2007 (DK) PA 2007 01054

(51) Int. Cl. C12N 5/071; C07K 17/10; C07K 16/00; B32B 5/00; C12N 15/63

(52) U.S. Cl. 435/375; 530/395; 530/391.7; 428/402; 435/320.1; 977/773; 977/795; 977/906

(57) ABSTRACT

The present invention relates to nanoparticles comprising osteopontin and a polymer carrier, preferably a cationic carrier. Preferably, the cationic carrier is chitosan. Osteopontin and/or the cationic carrier may have bioactivity and/or the nanoparticle may comprise an additional component with bioactivity. Such additional bioactive component may e.g. be a siRNA. The nanoparticles of the invention may be used for treatment of bone diseases or inflammatory diseases.

### BACKGROUND

Osteopontin is a multifunctional glycoprotein which is expressed by a wide variety of cell types including bone, smooth muscle, activated T-lymphocytes, macrophages, and carcinomas and sarcomas.

The protein is involved in a range of cellular functions including cell adhesion and spreading, cell migration and homing, chemotaxis, and calcium homeostasis (e.g., calcification).

In mammals, osteopontin is known to play an important role in regulation of bone formation and/or bone remodelling, regulation of immune responses, mediation of inflammation (e.g., tissue inflammation) in specific disease and injury states, angiogenesis, and arterial wound healing. Osteopontin has also been shown to have antibacterial effects.

Osteopontin binds to cells via integrin and non-integrin receptors. The presence of a Arg-Gly-Asp (RGD) cell-binding peptide sequence within the osteopontin molecule allows cell attachment and spreading via avβ3 integrins.

It has been shown that osteopontin when injected into mice accumulates in the bone tissue as a result of preferential cell-binding interaction with bone tissue cells.

A distinct receptor-ligand interaction between CD44 and osteopontin has also been shown to play a role in mediating chemotaxis and/or cell or attachment. In addition, differential attachment of osteoclasts to surfaces coated with osteopontin isolated from various tissues and to phosphorylated and nonphosphorylated osteopontin has been demonstrated.

Many therapeutic agents like osteopontin have not been utilised successfully because of their limited ability to reach the target tissue in therapeutically relevant amounts. New targeting delivery systems for anti-cancer agents, hormones, proteins, peptides and vaccines are necessary because of safety and efficacy problems with conventional naked systems. For example, cytotoxic cancer drugs can damage both malignant and normal cells due to non-specific accumulation. A drug delivery system that targets the drug to the malignant tumor would decrease toxicity. Both intravenous and oral administration of protein, RNA and DNA-based drugs typically result in degradation due to their instability in these environments. Additional problems include premature loss of efficacy due to rapid clearance and metabolism. Drug delivery systems that can deliver protein, nucleic acid or liable small molecules by overcoming extracellular and intracellular barriers are highly desirable and are currently the subject of ongoing research.

Polycationic polymer (polyplexes) and lipid-based (lipoplexes) and non-viral delivery systems are attractive candidates due to the immunogenicity and safety issues associated with viral delivery. A common strategy is to mix siRNA with a cationic polymer and lipid agent to form nanoscale polyplexes and lipoplexes.

Targeted delivery of nanoparticles can be achieved by either passive or active targeting. Active targeting of a therapeutic agent is achieved by conjugating the therapeutic agent or the carrier system to a tissue or cell-specific ligand. Passive targeting is achieved without targeting moieties due to passive accumulation of nanoparticles in the target organ. For example, drugs encapsulated in nanoparticles can passively target tumor tissue through the enhanced permeation and retention effect. This approach, however, is not as effective as a specific targeted strategy.

There has been considerable research into developing a controlled release of drugs at target sites. Depending on the method of preparation, nanoparticles can be obtained with different properties and release characteristics for the encapsulated therapeutic agents.

The advantages of using nanoparticles for drug delivery result from A) nanoparticles protect the therapeutic from chemical or enzymatic breakdown B) nanoparticles, because of their small size, can penetrate through smaller capillaries and are taken up by tissue cells which allow efficient drug accumulation at the target sites. In addition, the use of biodegradable polymers for nanoparticle preparation allows sustained drug release within the target site over a period of days, weeks or months.

### SUMMARY OF THE INVENTION

The invention provides a completely new nanoparticle system for drug delivery that is able to optimise and control the properties of osteopontin for a wide range of applications.

It has surprisingly been shown that osteopontin under curtain conditions is able to interact with a wide range of polycations (e.g. chitosan, PEI) in such a way that nanoparticles are formed. Furthermore, these specific nanoparticles can incorporate a wide range of therapeutic agents enabling the system to be directed against a wide range of diseases including osteoporosis, bone cancer, multiple sclerosis. The particles may also be used to inhibit bacterial growth (in solution and as biofilms) in the mouth, stomach and intestinal tract in animals and humans.

Thus, in a first aspect the present invention provides an osteopontin nanoparticle comprising osteopontin and a cationic carrier. A second aspect is use of the nanoparticle for medicine. A third aspect is a method of preparing the nanoparticle.

### BRIEF DESCRIPTION OF THE DRAWINGS

FIG. 1.

Osteopontin/chitosan particle formation: Z-average (in nm) in relation to the amount of osteopontin solution.

Varying amounts of 3 different 50 mg/mL osteopontin solutions:

Series 1: Standard osteopontin

Series 2: Full length (Pure)

Series 3: N-term part (exposed RGD-site)

(20 μL measurements were: 40753 nm, 56323 nm and 2469.7 nm.)

FIG. 2.

Osteopontin/chitosan particle formation: Polydispersity Index (PDI) in relation to osteopontin amount

(20 μL measurements were: 40753 nm, 56323 nm, 2469.7 nm.)

FIG. 3.

Osteopontin/siRNA/chitosan particle formation: size as a result of amount and order of addition.

1: Pink graph: Decreasing amount of osteopontin & increasing amount of siRNA. Osteopontin added first chitosan solution.

2: Blue graph: Increasing amount of osteopontin & decreasing amount of siRNA. siRNA added first to chitosan solution.

FIG. 4.

Uptake in osteoblasts vs. Hek293 after 1 hour transfection with cy3 labeled siRNA/osteopontin/chitosan nanoparticles.

Osteoblasts: in general very high uptake.

Hek293: in general no or very little uptake.

FIG. 5.

Uptake after 1 hour transfection of osteoblasts—cy3 labeled

siRNA/osteopontin/chitosan nanoparticles vs. cy3 labeled siRNA/chitosan nanoparticles.

FIG. 6.

Knockdown of GapDH in osteoblast.

If nothing else is mentioned, 1 hour transfection was performed.

Osteopontin=osteopontin/siRNA/chitosan particles

siRNA=siRNA/chitosan particles

mirus=TransIT-TKO/siRNA particles

### DISCLOSURE OF THE INVENTION

In a first aspect, the present invention provides an osteopontin nanoparticle comprising osteopontin and a polymer carrier. Preferably, the polymer carrier is a cationic carrier.

The term “osteopontin”, as used herein, relates to full-length human osteopontin, having the amino acid sequence that has been known since the late eighties. The term “osteopontin”, as used herein, further relates to any osteopontin derived from animals, such as murine, bovine (SEQ ID NO:1), or rat osteopontin, as long as there is sufficient identity in order to maintain osteopontin activity, and as long as the resulting molecule will not be immunogenic in humans. Bovine osteopontin is particular preferred.

The term “osteopontin”, as used herein, further relates to biologically active variants and fragments, such as the naturally occurring isoforms of osteopontin. Osteopontin is expressed in functionally distinct forms that differ at the level of transcription (alternative splicing) and posttranslational modifications (phosphorylation, glycosylation). Three splice variants of OPN (osteopontin) are known so far, designated OPN-a (herein also called “full-length” osteopontin), OPN-b and OPN-c.

A thrombin cleavage leads to two in vivo proteolytic cleavage fragments comprising the N- and C-terminal portions of the protein. Phosphorylation of osteopontin, in particular of the C-terminal portion of the proteins, may be important for osteopontin function. The term “osteopontin” as used herein, is therefore also meant to encompass these proteolytic fragments and differentially phosphorylated osteopontin forms.

The term “osteopontin”, as used herein, further encompasses isoforms, muteins, fused proteins, functional derivatives, active fractions or fragments, or circularly permutated derivatives, or salts thereof. These isoforms, muteins, fused proteins or functional derivatives, active fractions or fragments, or circularly permutated derivatives retain the biological activity of osteopontin. Preferably, they have a biological activity, which is improved as compared to wild type osteopontin.

Preferably, the osteopontin is obtained from milk, including naturally occurring fragments or peptides derived from OPN by proteolytic cleavage in the milk, or genesplice-, phosphorylation-, or glycosylation variants as obtainable from the method proposed in WO 01/49741. The milk can be milk from any milk producing animals, such as cows, camels, goats, sheep, dromedaries and llamas. However, OPN from bovine milk is preferred due to the availability. OPN or derivates thereof can also be genetically prepared.

Preferred amino acid sequence of osteopontin (SEQ ID NO:1):

LPVKPTSSGSSEEKQLNNKYPDAVAIWLKPDPSQKQTFLAPQNSVSSEE
TDDNKQNTLPSKSNESPEQTDDLDDDDDNSQDVNSNDSDDAETTDDPDHS
DESHHSDESDEVDFPTDIPTIAVFTPFIPTESANDGRGDSVAYGLKSRSKK
FRRSNVQSPDATEEDFTSHIESEEMHDAPKKTSQLTDHSKETNSSELSKE
LTPKRKDKNKHSNLIESQENSKKLSQEFHSLEDLDLDHKSEEDKHL
KIRISHELDSASSEVN

Nanoparticles comprising osteopontin has various beneficial characteristics.

Osteopontin has an inhibitory effect on inflammatory diseases such as multiple sclerosis and improved delivery of osteopontin is beneficial for treatment of inflammatory diseases. Examples include inhibition of bacterial growth and biofilm formation on teeth and oral cavity, antibiotic effects in lung, skin, stomach and intestine. The immunoregulatoy effects of osteopontin in particulate form may also be exploited in treatment of inflammatory diseases (e.g. rheumatoid arthritis and Crohn's disease) and wound heeling.

Osteopontin also been demonstrated to inhibit bacterial growth, wherefore the nanoparticles of the invention may be used for treatment of bacterial infections.

Moreover, osteopontin inhibits plague formation on teeth. Thus, the nanoparticles of the invention may be used for dental applications.

The nanoparticles may be systemically delivered or delivered locally from an implant. I.e. the nanoparticles have possible applications in tissue engineering (biocompatibility) and controlling foreign body responses.

In a preferred embodiment of the invention, the other constituent of the osteopontin nanoparticle is a polymer carrier selected from the group consisting of polyethyleneimine (PEI), poly (lysine) (PLL), poly(2-dimethyl-amino)ethyl methacrylate (pDMAEMA), chitosan, histidine-based polypeptides, poly(lactic acid) (PLA), polylactide/glycolide acid co-polymers (PLGA), poly(lacticglycolide) acid, polyethylene glycol (PEG), and poly[N-(2-hydroxpropyl)methacrylamide] (PHPMA).

More preferably, the polymer carrier is a cationic carrier and preferably, the cationic carrier is selected from the group consisting of polyethyleneimine (PEI), poly (lysine) (PLL), poly(2-dimethyl-amino)ethyl methacrylate (pDMAEMA), chitosan and histidine-based polypeptides.

Most preferably, the cationic carrier is chitosan. Chitosan can improve delivery of osteopontin to mucosal surfaces e.g. reinforce osteopontins inhibitory effect of plague formation, by sticking to the mouth mucosa.

The weight ratio (w:w) of chitosan and osteopontin in the nanoparticle is typically less than 100. More preferably, the ratio is less than 10 and even more preferred the ratio is between 1 and 5. The most monodisperse particles are obtained at weight ratio just around 1 to 0.5—further decreasing the ratio results in aggregation. The smallest particles are obtained at weight ratio of around 1.

The size of the osteopontin nanoparticle may be between 10 and 1000 nm, but it is preferred that the size is between 50 nm and 500 nm. This size range is obtainable at a weight ratio below 10 and results in the highest uptake.

In addition to the above mentioned characteristics, osteopontin has the ability of targeting the nanoparticle to bone tissue. Thus, the nanoparticles of the invention can be used for treatment of conditions in bone tissue, e.g. bone cancer, osteoporosis.

In a preferred embodiment, the osteopontin nanoparticle further comprises an additional bioactive component. When referring to an additional bioactive component, it is implied that osteopontin and/or the cationic carrier also may have bioactivity.

The additional bioactive component may be selected from the group of an antibody, an aptamer, a siRNA, a microRNA, microRNA inhibitory antisense oligonucleotidean antisense oligonucleotide, preferably activating RNase H, a plasmid, a small molecule, polyethylene (glycol) (PEG) and HPMA and cationic co-polymers of PEG and HPMA. Typically, the bioactive component has already been described and it is desired to improve the delivery of the bioactive component. For example the nanoparticle can be used to deliver RNA-based gene silencing therapeutics such as siRNA for RNA interference. Stealth coatings composed of PEG or PHPMA may enable prolonged circulation of the nanoparticle.

When the cationic carrier is chitosan, it is preferred that the chitosan has a deacetylation degree of at least 60% and a molecular weight of at least 10 kDa.

In another embodiment, the chitosan has a molecular weight selected from the group consisting of at least 10 kDa, at least 20 kDa, at least 30 kDa, at least 40 kDa, at least 50 kDa, at least 75 kDa and at least 100 kDa. Preferably, the chitosan has a molecular weight of no more than 500 kDa.

A second aspect of the invention is the osteopontin nanoparticle of the invention for use as medicine.

A third aspect of the invention is the nanoparticle of the invention for the preparation of a medicament for treatment of bone disease, inflammatory diseases, bacterial infections or dental diseases.

The bone disease may be bone cancer and osteoporosis; the inflammatory disease may be arthritis.

A fourth aspect of the invention is method of preparing an osteopontin nanoparticle comprising osteopontin and a cationic carrier comprising
a. Providing a osteopontin solution
b. Providing a solution comprising a cationic carrier
c. Mixing the solution of step a with the solution of step b

In a preferred embodiment, the method further comprises adding an additional bioactive component to the solution of step a, step b or step c.

Preferably, the additional bioactive component is an RNA-based gene silencing therapeutics e.g. siRNA.

In a preferred embodiment, the cationic carrier is chitosan.

### EXAMPLES

### Example 1

### Preparation of Particles

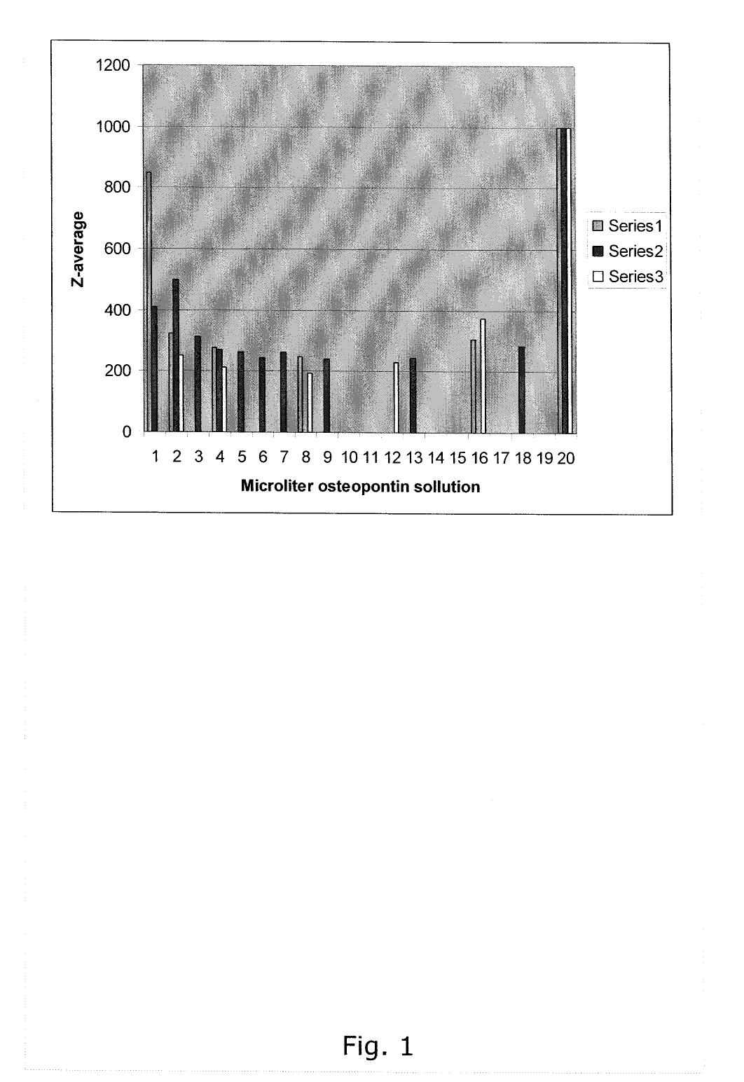

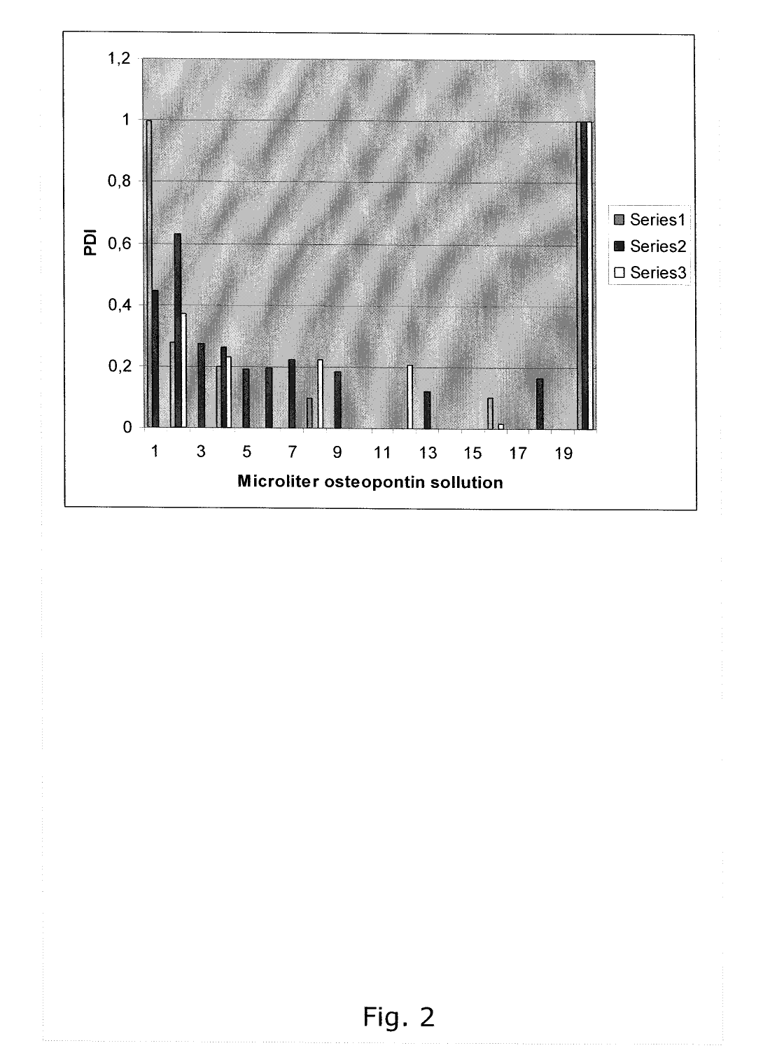

High molecular weight chitosan is dissolved in sodium acetate buffer pH 4.5 to a concentration of 1 mg/ml. 800 μL chitosan solution and 200 μL acetate buffer is mixed in a reaction tube and 1-35 μL of a 50 mg/mL osteopontin solution is added while stirring constantly for 1 hour. This creates monodisperse nanosize particles in the range of 100-500 nm. (FIGS. 1 and 2)

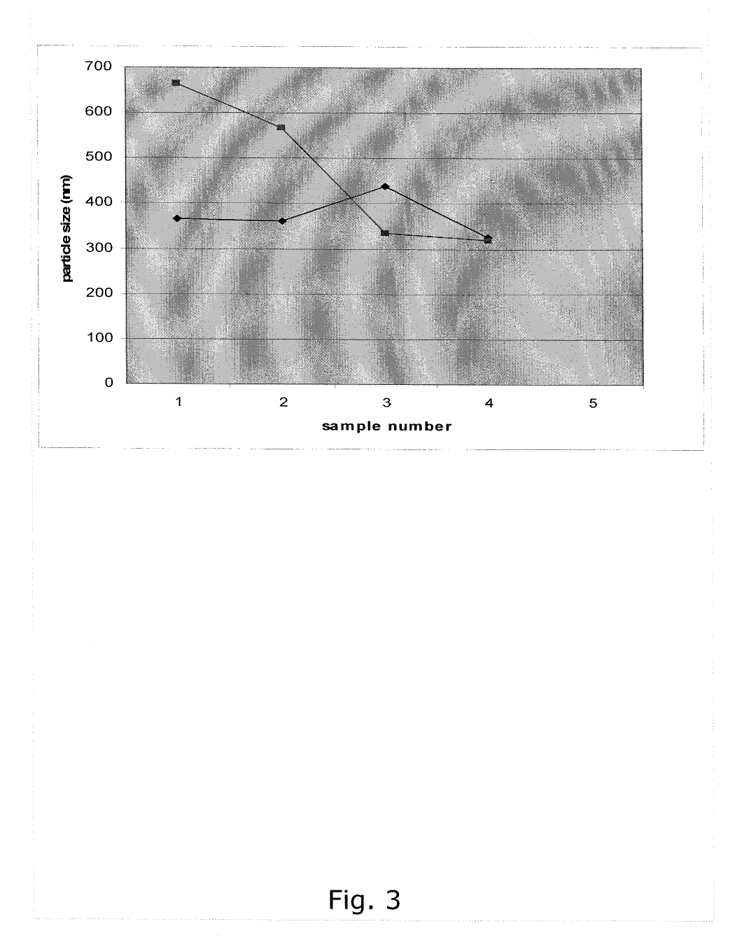

If other therapeutics or targeting ligands needs to be incorporated, these are added to the chitosan solution either prior to or subsequently to addition of osteopontin. In regards to siRNA we add 20 μL of 20 μM solution 10 min prior to addition of osteopontin. (FIG. 3)

The presence of osteopontin in the particles increases the stability of the particles without decreasing transfection abilities. A high stability is crucial for any drug delivery system to overcome extra cellular barriers in the organism.

### Example 2

### Increased Uptake and Knockdown in Osteoblasts

Experiments with osteopontin/chitosan/siRNA-nanoparticles has shown increased uptake and knockdown in osteoblasts in comparison to traditional chitosan/siRNA-nanoparticles.

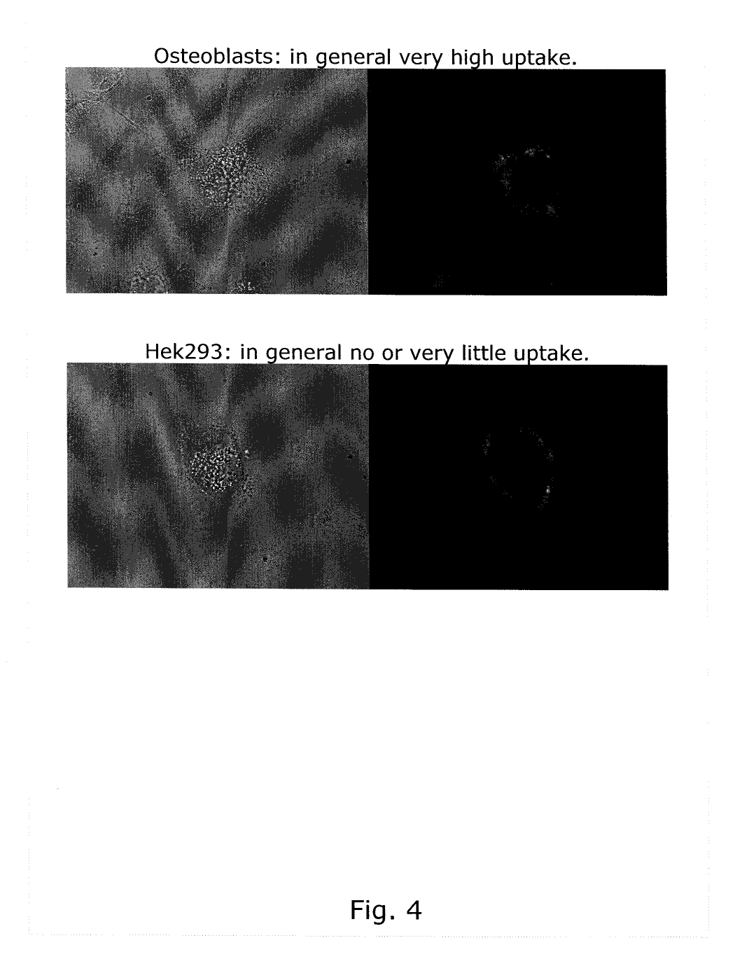

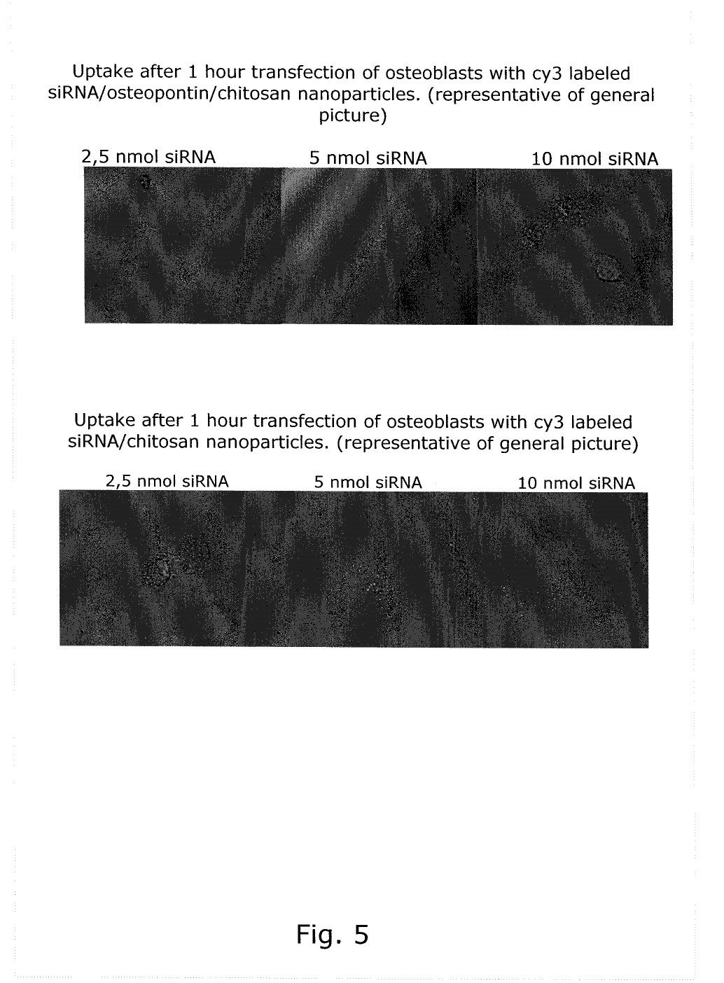

The uptake experiments were performed in 6-well plates with coverslips and analysed with confocal microscopy. A clear increase in uptake was observed in osteoblasts when comparing osteopontin/chitosan/siRNAnanoparticles to chitosan-siRNA-nanoparticles. (FIGS. 4 and 5)

GapDH knockdown experiments show a clear increase in the particles ability to silence genes in comparison to siRNA/chitosan nanoparticles and especially in comparison to commercial TransIT-TKO/siRNA particles.

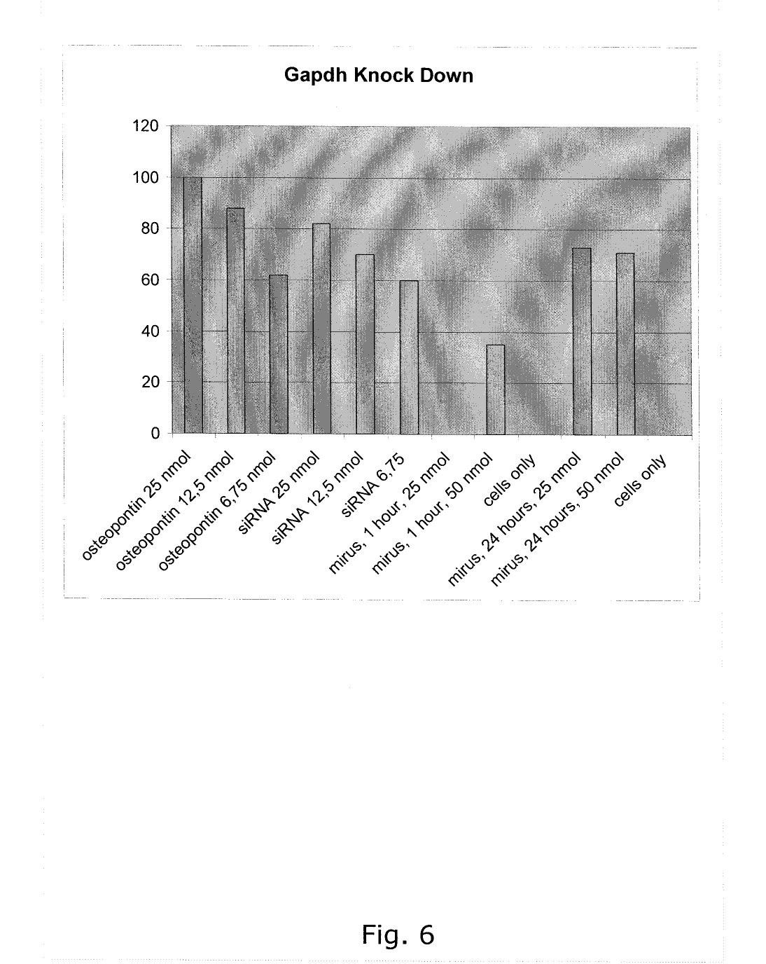

The experiment was performed in 6-well plates with a siRNA concentration of 25 nm. The cells were harvested after 48 hours and the RNA was isolated using trizol. The same amount of RNA from each sample was run on a 1×MOPS agarose gel with 1×MOPS as running buffer. A blot was performed to transfer the RNA to a membrane. After prehybridation with salmon sperm DNA the membrane was subjectet to a radioactive gapDH oligo probe. (FIG. 6)

It is clear that the presence of osteopontin in a nanoparticle increases the uptake of this particle into osteoblasts, thereby increasing the potential knockdown. In the organism this ability results in a targeting system with preferential uptake in bone tissue. The increased stability of the particles further increases the circulation time of the particles increasing the possibility of reaching the target tissue.

1. An osteopontin nanoparticle comprising osteopontin and a polymer carrier.

2. The osteopontin nanoparticle of claim 1, wherein the polymer carrier is selected from the group consisting of polyethyleneimine (PEI), poly (lysine) (PLL), poly(2-dimethyl-amino)ethyl methacrylate (pDMAEMA), chitosan, histidine-based polypeptides, poly(lactic acid) (PLA), polylactide/glycolide acid co-polymers (PLGA), poly(lactic-glycolide) acid, polyethylene glycol (PEG) and poly[N-(2-hydroxpropyl)methacrylamide] (PHPMA).

3. The osteopontin nanoparticle of claim 1, wherein the polymer carrier is a cationic carrier selected from the group consisting of polyethyleneimine (PEI), poly (lysine) (PLL), poly(2-dimethyl-amino)ethyl methacrylate (pDMAEMA), chitosan and histidine-based polypeptides.

4. The osteopontin nanoparticle of claim 3, wherein the cationic carrier is chitosan.

5. The osteopontin nanoparticle of claim 4, wherein the weight ratio of chitosan and osteopontin is between 1:1 and 1:5.

6. The osteopontin nanoparticle of claim 1, wherein the size of the particle is between 50 and 500 nm.

7. The osteopontin nanoparticle of claim 1, further comprising an additional bioactive component.

8. The osteopontin nanoparticle of claim 7, wherein the bioactive component is selected from the group consisting of an antibody, an aptamer, an siRNA, a microRNA, a microRNA inhibitor, an antisense oligonucleotide, a plasmid and a small molecule.

9. The osteopontin nanoparticle of claim 4, wherein the chitosan has a deacetylation degree of at least 60% and a molecular weight of at least 10 kDa.

10. The osteopontin nanoparticle of claim 1, wherein said osteopontin nanoparticle is formulated for uptake by osteoblasts.

11. (canceled)

12. A method of preparing an osteopontin nanoparticle comprising:
a. providing an osteopontin solution;
b. providing a solution comprising a cationic carrier; and
c. mixing the solution of step a with the solution of step b.

13. The method of claim 12 further comprising adding an additional bioactive component to the solution of step a, step b or step c.

14. The method of claim 13, wherein the bioactive component is a siRNA.

15. The osteopontin nanoparticle of claim 4, further comprising an additional bioactive component.

16. The osteopontin nanoparticle of claim 15, wherein the bioactive component is a siRNA.

17. A method of delivering a nucleic acid to an osteoblast comprising:
providing an osteopontin nanoparticle that comprises osteopontin, chitosan, and a nucleic acid; and
contacting an osteoblast with said osteopontin nanoparticle under conditions sufficient for uptake of said nucleic acid by said osteoblast.
